(19) United States
(12) Patent Application Publication
(10) Pub. No.: US 2008/0292669 A1
(43) Pub. Date: Nov. 27, 2008

(54) Foamed Substrate and Method for Making Same

(75) Inventors: Fang Deng, Parsippany, NJ (US); Stanley J. Lech, Parsippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLNE BEECHAM CORPORTION

(21) Appl. No.: 12/094,650

(22) PCT Filed: Nov. 20, 2006
(86) PCT No.: PCT/US06/61090

§ 371 Date: May 22, 2008

Related U.S. Application Data
(60) Provisional application No. 60/738,923, filed on Nov. 22, 2005.

(51) Int. Cl. A61K 8/72; A61K 6/02; A61Q 11/00

(52) U.S. Cl. 424/401; 523/120; 424/49

(57) ABSTRACT

The present invention provides a foamed and/or aerated substrate for transmucosal and/or transdermal applications. The foamed and/or aerated substrate may be in the form of a film, sheet, strip or wafer. The foamed and/or aerated substrate is suitable for use in applications such as, for example, adhesive denture liners, tooth whitening, and smoking control/cessation. The foamed and/or aerated substrate is polymeric in nature and comprises one or more polymers and one or more foaming agents. Additionally, the foamed and/or aerated substrate may include, but not limited to, one or more plasticizers, hydrophobic barrier agents, tooth whitening agents, antioxidants, preservatives, super-disintegrants or absorbents, flavorants, deodorants, breath freshening agents, colorants, surfactants, film modifiers, cross-linking agents, antimicrobial agents, control release agents, other therapeutic agents, or any combinations thereof.

### BACKGROUND OF THE INVENTION

1. Field of the Invention

The invention is directed to foamed substrates. More particularly, the present invention is directed to foamed and/or aerated films for transmucosal and transdermal applications.

2. Description of Related Art

The use of polymer-based transmucosal and/or transdermal films to deliver one or more active components is known in the art. Several drawbacks are associated with these films, including but not limited to, rigidity, excessive weight, undesirable aesthetics and discomfort to users.

Despite the progress that has been made in the use of polymer-based transmucosal and/or transdermal films to deliver one or more active components the search for more effective materials continues. There is still a strong need for a film that is both strong and effective enough to withstand use in a transmucosal and/or transdermal environment while being conformable to a user during use. The present invention meets and/or exceeds this need.

### SUMMARY OF THE INVENTION

The principal object of the present invention therefore is to provide foamed and/or aerated films for transmucosal and/or transdermal applications.

It is another object of the present invention to provide a foamed and/or aerated film that is strong and highly conformable.

It is yet another object of the present invention to provide a foamed and/or aerated film that is lightweight and quickly hydratable.

It is a further object of the present invention to provide such a foamed and/or aerated film that functions as an adhesive denture liner.

It is a still a further object of the present invention to provide such a foamed and/or aerated adhesive film that functions as a tooth whitening strip.

It is still a further object of the present invention to provide such a foamed and/or aerated film that is self-supporting, nontoxic and organoleptically acceptable.

Additional objects and advantages of the invention will be set forth in part in the description that follows, and in part will be obvious from this description, or may be learned by practice of the invention. The objects and advantages of the invention may be realized and attained by means of the instrumentalities and combinations particularly pointed out in the application.

To achieve the foregoing objects and following the purpose of the invention, as embodied and broadly described herein, the invention provides a foamed and/or aerated film formed from one or more polymeric materials and one or more foaming agents.

To further achieve the foregoing objects and in accordance with the purpose of the invention, the invention further provides a method for making a foamed and/or aerated film.

### DETAILED DESCRIPTION OF THE INVENTION

The present invention provides a foamed and/or aerated substrate for transmucosal and/or transdermal applications. The foamed and/or aerated substrate may be in the form of a film, sheet, strip, wafer, or a rope- or string-like form. These forms of the foamed and/or aerated substrate enable the product to be conveniently packaged and torn into desirable lengths by the consumer prior to application. The foamed and/or aerated substrate is suitable for use in applications such as, for example, adhesive denture liners, tooth whitening, smoking control/cessation, and the like.

It should be understood that the term “foamed”, as used hereinafter in this application, is meant to include foamed and/or aerated.

The foamed substrate is polymeric in nature and has one or more polymers and one or more foaming agents. Additionally, the foamed substrate may include, but is not limited to, one or more plasticizers, hydrophobic barrier agents, tooth whitening agents, antioxidants, preservatives, super-disintegrants or absorbents, flavorants, deodorants, breath freshening agents, colorants, surfactants, film modifiers, cross-linking agents, antimicrobial agents, control release agents, other therapeutic agents, or any combinations thereof.

To best fit consumer's usage experience of these materials, the foamed article should possess a desirable structure in terms of foam density, percent of open and closed cells, cell size, cell geometry, orientation of cell walls, or any combinations thereof. The foamed structure should also have enough thermal and environmental stability during transportation and storage. The foamed material may be fabricated into various desired shapes and packaged by various different ways.

In a first embodiment of the present invention, a denture adhesive liner in the form of a foamed or an aerated extruded film or sheet, strip or wafer is provided. These films or strips will possess a certain desirable thickness, strength and integrity during their application. Upon hydration by saliva or during actual use in the oral environment, the material or product hydrates and becomes tacky and develops adhesiveness between muco tissues and denture.

In a second embodiment of the present invention, a denture adhesive liner in the form of a foamed and/or aerated extruded rope- or string-like form is provided. By formatting the liner in this form, the consumer can dispense a desirable length of the product, tear it from the dispenser and apply it as appropriate.

The materials used for preparing such an extruded film or sheet can include but are not limited to: any water-soluble, water swellable, water dispersible or water insoluble polymer suitable for use as an active ingredient for a denture adhesive; reactive and non-reactive foaming agent; hydrophobic barrier agent; plasticizer; antioxidant; preservative; super-disintegrant or absorbent; flavorant; deodorant and breath freshening agent; colorant; surfactant; film modifier; cross-linking agent; antimicrobial agent; control release agent; therapeutic agent; or any combinations thereof.

The foamed denture adhesive liner may include any suitable denture adhesive material. Examples include, but are not limited to, one or more polyvinyl alcohols, polyethylene oxides, karaya gum, methyl vinyl ether/maleic anhydride copolymers and hydrated derivatives thereof and partial salts thereof, sodium alginate, or any combinations thereof.

The foamed denture adhesive liner according to the present invention also includes one or more thermoplastic polymers. The one or more thermoplastic polymers are water-soluble. A “thermoplastic polymer” is meant to refer to a material, which is melt processable. As used herein, the term “thermoplastic” refers to a material which softens and/or becomes flexible when exposed to heat and generally returns to its original condition when cooled to room temperature.

As used herein, a material will be considered to be “water-soluble” when it substantially dissolves in excess water to form a solution, thereby losing its initial form and becoming essentially molecularly dispersed throughout the water solution. Typically, the water-soluble material will be free from a substantial degree of cross-linking, as cross-linking tends to render a material water insoluble. Also used herein, the term “water-insoluble” is meant to refer to a material that, when exposed to an excess of water, disperses but does not dissolve. As such, a water-insoluble material generally retains its original identity or physical structure, but in a highly dispersed state and must have sufficient physical integrity to resist flow and fusion with neighboring materials.

Suitable thermoplastic polymers for use in the present invention include, but are not limited to, polyethylene oxide polymer, hydroxypropylcellulose, hydroxypropylmethyl-cellulose, or any combinations thereof.

Hydroxypropylcellulose (“HPC”) polymers having a weight average molecular weight between 80,000-1,150,000 are useful for the purposes of this invention. HPC can be used as a thermoplastic polymer component by itself or in combination with other thermoplastic polymer components, i.e., PEO, and the like. HPC is commercially available from Hercules, Inc. (Wilmington, Del.) under the trade name KLUCEL. In one embodiment, HPC is present in the denture adhesive liner composition of the present invention in an amount between about 0 wt. % and about 90 wt. %, based on the total weight of the composition. In another embodiment, HPC is present between about 5 wt. % and about 20 wt. % in combination with another thermoplastic polymer. In yet a third embodiment, HPC is present as the only thermoplastic polymer in an amount between about 60 wt. % and about 90 wt. %.

Hydroxypropylmethylcellulose (“HPMC”) is another water-soluble cellulose that exhibits thermoplastic polymer processing properties when used in combination with a plasticizer. HPMC can be used as a thermoplastic polymer component by itself or in combination with other thermoplastic polymer components, i.e., PEO, and the like. HPMC is commercially available from Dow Chemical Company of Midland, Mich., USA, under the trade name METHOCEL, which is an HPMC, that has a 2% concentration in water and produces a viscosity of 400 cPs.

In one embodiment, HPMC is present in the denture adhesive liner composition of the present invention in an amount between about 0 wt. % and about 90 wt. %, based on the total weight of the composition. In another embodiment, HPMC is present between about 5 wt. % and about 20 wt. % in combination with another thermoplastic polymer. In yet a third embodiment, HPMC is present as the only thermoplastic polymer in an amount between about 60 wt. % and about 90 wt. %.

If polyethylene oxide polymer (“PEO”) is used, it is desirable that the material exhibits a weight average molecular weight that is effective for the denture adhesive liner composition to exhibit sufficient cohesive strength and resistance to degradation properties. In general, if the weight average molecular weight of a PEO polymer is too high, the polymer chains may become heavily entangled which may result in a thermoplastic composition, which is difficult to process. In one embodiment, the PEO polymers suitable for use in the present invention exhibit a weight average molecular weight between about 100,000 to about 20,000,000. In another embodiment, the weight average molecular weight is between about 200,000 to about 8,000,000.

In one embodiment, PEO is present in the foamed denture adhesive liner composition of the present invention in an amount between 0 and 90 wt. %. In another embodiment, PEO is present in an amount between 50 and 90 wt. %. In a third embodiment, PEO is present between 30 and 70 wt. % in combination with another thermoplastic polymer. In yet a fourth embodiment, PEO is present as the only thermoplastic polymer in an amount between about 30 wt. % and about 90 wt. %.

It is generally desired that a toxicologically acceptable compatibilizer or plasticizer be used as an optional third component in an amount sufficient for the denture adhesive composition to exhibit desired extrusion processability properties. The term “toxicologically acceptable”, as used herein, describes materials which are suitable in their toxicity profile for administration to humans and/or lower animals.

Suitable plasticizers include, but are not limited to, water; polyethylene oxide; polypropylene oxide; one or more glycols such as propylene glycol and polyethylene glycol; one or more polyhydric alcohols such as glycerin and sorbitol; one or more glycerol esters such as glycerol triacetate; one or more fatty acid triglycerides; one or more naphthenic oils; one or more aromatic oils; one or more vegetable oils such as castor oil; one or more low molecular weight rosin esters, one or more polyterpenes, or any combinations thereof.

If HPMC is used as a thermoplastic polymer by itself, propylene glycol may be used as a plasticizer during melt processing. If PEO is used as a thermoplastic polymer by itself, it has been suggested that water may be used as a fugitive plasticizer for PEO during melt processing.

Plasticizer may be present in an amount of about 1 wt. % to about 30 wt. %, based on the total weight of the composition. In one embodiment, plasticizer is included in an amount of about 5 wt. % to about 25 wt. %, and in yet another embodiment about 3 wt. % to about 6 wt. %.

Optionally, the foamed denture adhesive liner composition may comprise one or more therapeutic actives suitable for mucosal or topical administration. The phrase “suitable for mucosal or topical administration”, as used herein, describes agents which are pharmacologically active when absorbed through internal mucosal surfaces of the body such as the oral cavity, or applied to the surfaces of the skin. Therapeutic actives may be present at a level of from about 0 wt. % to about 30 wt. %, based on the total weight of the composition.

Therapeutic actives that are useful in the compositions of the present invention include, but are not limited to, one or more antimicrobial agents; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, cimetidine, eugenol, or hydrocortisone; dentinal desensitizing agents such as potassium nitrate, potassium chloride, strontium chloride or sodium fluoride; anesthetic agents such as lidocaine or benzocaine; anti-fungals; aromatics such as camphor, eucalyptus oil, and aldehyde derivatives such as benzaldehyde; insulin; steroids; and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

Suitable antimicrobial agents for use in the compositions of the present invention may include, but are not limited to, one or more of cetylpyridium chloride, domiphen bromide, iodine, sulfonamide, bisbiguanide, phenolic, benzoic acid, sodium benzoate, paraben, sorbic acid, sorbate, propionic acid, propionate, acetic acid, acetate, nitrate, nitrite, sulfur dioxide, sulfite, antibiotic, diethyl pyrocarbonate, epoxide, hydrogen peroxide, phosphate, or any combinations thereof.

The one or more antimicrobial agents may be provided in an amount that provides effective antimicrobial properties to the composition. The one or more antimicrobial agents may be present in an amount about 0.0001% to about 2.0% by total weight of the composition. In one embodiment, the one or more antimicrobial agents are present in an amount about 0.001% to about 1.0% by weight, and in another embodiment from about 0.01% to about 0.5% by weight of the composition.

The compositions of the present invention may include one or more odor adsorbent components. Suitable odor adsorbent components include, but are not limited to, cyclodextrin, activated carbon, sodium bicarbonate, or any combinations thereof. Suitable cyclodextrins for use in the present invention include, but are not limited to, one or more α-cyclodextrins, β-cyclodextrins, γ-cyclodextrins, carboxylmethyl cyclodextrins, hydroxypropyl cyclodextrins, methyl cyclodextrins, sulfated cyclodextrins, quaternary ammonium cyclodextrins, or any combinations thereof.

The one or more odor adsorbent components may be present in an amount about 0 wt. % to about 30 wt. %.

The composition may also comprise one or more additional suitable components including, but not limited to, silicon dioxide, antioxidant such as ascorbic acid, colorant, preservative such as methyl and propyl paraben, thickener, flavor, fragrance, sensate enhancer, or any combinations thereof.

Suitable components that provide flavor, fragrance, and/or sensate benefit include, but are not limited to, natural or artificial sweetening agents, menthol, menthyl lactate, wintergreen oil, peppermint oil, spearmint oil, leaf alcohol, coolant such as 3-menthoxypropane-1,2-diol and paramenthane, and carboxyamide agent such as N-ethyl-menthane-3-carboxamide.

The one or more additional suitable components may be present in the compositions of the present invention in an amount about 0 wt. % to about 20 wt. %, based on the total weight of the composition.

In a second embodiment of the present invention, a foamed tooth whitening strip is provided. The materials used for preparing such foamed tooth whitening strip can include but is not limited to: any water-soluble, water swellable, water dispersible or water insoluble polymer suitable for use as a tooth whitening strip; whitening agent; reactive and non-reactive foaming agent; hydrophobic barrier agent; plasticizer; antioxidant; preservative; super-disintegrant or absorbent; flavorant; deodorant and breath freshening agent; colorant; surfactant; film modifier; cross-linking agent; antimicrobial agent; control release agent; therapeutic agent; or any combinations thereof.

In one embodiment according to the present invention, the whitening strip of the present invention has a polymer system that includes a first polymer, namely water-soluble or water dispersible poly-(vinylpyrrolidone) (PVP) or any of its derivatives, and at least one second polymer. The combination of the first and second polymers define the water-soluble and/or water swellable and/or water dispersible polymer system. The polymer system has adhesive properties, such that when it is brought to the teeth, the strip will adhere to the teeth.

The second polymer that can be used with the first polymer to produce the water-soluble or water dispersible polymer system according to the present invention, includes one or more of: an alkyl vinyl ether/maleic anhydride copolymer, alkyl vinyl ether/maleic acid copolymer, alkali metal or an amine salt of alkyl vinyl ether/maleic acid copolymer, partially or fully cross-linked alkyl vinyl ether/maleic anhydride copolymer, vinyl acetate copolymer, polyacrylates, polyurethane interpolymers, chitosan, poly(acrylic acid), poly(vinyl alcohol), poly(vinyl alcohol-g-ethylene glycol) copolymer, cellulose derivative, hydroxypropyl-methyl cellulose, hydroxyl-ethyl cellulose, hydroxypropyl cellulose, poly(ethylene oxide), poly(propylene oxide), Polyquaterium-11, Polyquaterium-39, poloxamer, carbomer, gelatin, starch, alginic acid, salt of alginic acid, natural gums such as gum karaya, xanthan gum, guar gum, arabic gum, tragacanth, or any combinations thereof.

In one embodiment of the present invention, the combination of the first and second polymers is one or more of the following: a poly(vinylpyrrolidone)-alkyl vinyl ether/maleic anhydride copolymer; poly(vinylpyrrolidone)-alkyl vinyl ether/maleic acid copolymer; poly(vinylpyrrolidone)-alkali metal or an amine salt of alkyl vinyl ether/maleic acid copolymer; poly(vinylpyrrolidone)—partially or fully cross-linked alkyl vinyl ether/maleic anhydride copolymer; poly(vinylpyrrolidone)-vinyl acetate copolymer; poly(vinylpyrrolidone)-polyurethane interpolymer; poly(vinylpyrrolidone)-chitosan; poly(vinylpyrrolidone)-polyacrylates; poly(vinylpyrrolidone)-poly(acrylic acid); poly(vinylpyrrolidone)-poly(vinyl alcohol); poly(vinylpyrrolidone)-poly(vinyl alcohol-g-ethylene glycol) copolymer; poly(vinylpyrrolidone)-cellulose derivative; poly(vinylpyrrolidone)-hydroxypropylmethyl cellulose; poly(vinylpyrrolidone)-hydroxyethyl cellulose; poly(vinylpyrrolidone)-hydroxypropyl cellulose; poly(vinylpyrrolidone)-poly(ethylene oxide); poly(vinylpyrrolidone)-poly(propylene oxide); poly(vinylpyrrolidone)-Polyquaterium-11; poly(vinylpyrrolidone)-Polyquaterium-39; poly(vinylpyrrolidone)-poloxamer; poly(vinylpyrrolidone)-carbomer; poly(vinylpyrrolidone)-gelatin; poly(vinylpyrrolidone)-starch; poly(vinylpyrrolidone)-alginic acid; poly(vinylpyrrolidone)-salt of alginic acid; poly(vinylpyrrolidone)-gum karaya; poly(vinylpyrrolidone)-xanthan gum; poly(vinylpyrrolidone)-guar gum; poly(vinylpyrrolidone)-arabic gum; poly(vinylpyrrolidone)-tragacanth; or any combinations thereof.

Commercial examples of such a first polymer include, but are not limited to, poly alkyl vinyl ether-maleic acid copolymer (PVM/MA copolymer), such as, Gantrez AN 119, AN 139, S-97 and MS-955, poly(vinyl alcohol); poly(acrylic acid); Poloxamer 407 (Pluronic); poly(vinyl-pyrrolidone-co-vinylacetate) copolymer (PVP/VA copolymer), such as Luviskol VA and Plasdone S630 PVP/VA; poly(vinylpyrrolidone) (PVP, K-15 to about K-120); Polyquaterium-11 (Gafquat 755N); Polyquaterium-39 (Merquat plus 3330); carbomer (Carbopol); hydroxypropylmethyl cellulose; hydroxyl-ethyl cellulose; hydroxypropyl cellulose; gelatin; and alginate salt such as sodium alginate. In one embodiment of the present invention, the polyurethanes include polycarbamyl polyglycol esters and poly(vinylpyrrolidone-polyurethane) interpolymer, such as, a poly(vinylpyrrolidone/polycarbamyl) polyglycol ester interpolymer.

The combination of the first and second polymers is a combination of PVP and another polymer. The polymers can favorably interact or complex to effectively increase the molecular weight of the polymer mixture. A linking agent can also be incorporated to promote the interaction between polymers. The effective increase in molecular weight results in a decrease in the rate of dissolution of the strip. Without being bound by any theory or structure, it is believed that the interaction between PVP and the polymers described in the examples, including polymers, such as, Gantrez, Aquamere, Chitosan and Pecogel, is accomplished through hydrogen bonding. Such interactions enable the polymer complex to bind onto the teeth and thereby provide effective whitening of the teeth.

In one embodiment, the water-soluble or water dispersible polymer system is present up to about 99.9 wt % based on the total weight of the whitening strip. In yet another embodiment, the polymer system is about 60 wt % to about 98 wt % of the total weight of the whitening strip.

Also, the amount of the first polymer is about 1 wt % to about 99 wt. %. In another embodiment the amount of the first polymer is about 25 wt. % to about 75 wt %, of the total weight of the whitening strip. The amount of the second polymer is about 1 wt % to about 80 wt %, and in another embodiment about 5 wt % to about 50 wt %, of the total weight of the whitening strip.

The whitening agent, which is an active agent, in the strip can, upon contact with saliva, release the active agent onto the teeth in the oral cavity. Alternatively, the active agent can permeate through the film and be released to the surface where it is applied, including surfaces, such as, enamel, gum tissue and tongue.

The whitening agents that can be used in the present invention include hydrogen peroxide; carbamide peroxide; peroxycarbamate; persulfate, such as, persulfate salt or percarbonate salt; a perboric acid; perborate salt; PVP-hydrogen peroxide complex; calcium peroxide; metal chlorite (e.g. calcium chlorite, barium chlorite, magnesium chlorite, lithium chlorite, sodium chlorite, and potassium chlorite), hydroperoxide; peroxyacids; organic peroxides (e.g., benzoyl peroxide) chlorine dioxide; hydrogen peroxide adduct of carbodiimide persulfate; peroxide-generating compounds (e.g., azobisisobutyronitrile), phosphates, polyphosphates, alkali metal phosphates, alkali metal polyphosphates, sodium tripolyphosphate, or any combinations thereof.

The whitening agent is present up to about 99 wt. % based on the total weight of the whitening strip. In one embodiment, the whitening agent is about 0.5 wt. % to about 99 wt. % of the total weight of the whitening strip. In yet another embodiment, the whitening agent is about 2 wt. % to about 75 wt. % of the total weight of the whitening strip.

In one embodiment of the present invention, the whitening agent is an alkali metal polyphosphate, such as sodium tripolyphosphate, and it is present in an amount about 7.5 wt. % to about 25 wt. % of the total weight of the whitening strip.

The whitening strip can also have an ingredient that further enhances benefits to the oral cavity and teeth. Such ingredients include: an antimicrobial agent, a mineralization compound, a stain prevention compound, a desensitization compound, an anti-calculus agent, a flavoring agent, an anti-inflammatory agent, an antioxidant, a volatile sulfur scavenger, an odorant neutralizer, and/or a vitamin. The whitening strip may also have a penetration enhancer, a plasticizer, a preservative, a surfactant or wetting agent, an anesthetic, an anti-allergenic, a pharmaceutical, or any combinations thereof. However, in an embodiment of the present invention, the whitening strip is free of surfactants that are undesired in the oral cavity.

Antimicrobial agents that can be used in the present whitening strip include, but are not limited to, polyphenol (e.g. triclosan) zinc salt, stannous fluoride, chlorhexidine, hexetidine, sanguinarine, benzalkonium chloride, salicylanilide, domiphen bromide, cetylpyridinium chloride, tetradecylpyridinium chloride (TPC), N-tetradecyl-4-ethylpyridinium chloride (TDEPC), octenidine, delmopinol, octapinol, and other piperidine derivatives, nicin preparation, zinc/stannous ion agent, antibiotics such as augmentin, amoxicillin, tetracycline, doxycycline, minocycline, and metronidazole, and analogs and salts of the above, essential oils including thymol, menthol, eugenol, geraniol, carvacrol, citral, hinokitiol, eucalyptol, catechol, methyl salicylate, hydrogen peroxide, metal salts of chlorite, or any combinations of all of the above.

Mineralization compounds that can be used in the present whitening strip include, but are not limited to, sodium monofluorophosphate, potassium monofluorophosphate, magnesium monofluorophosphate, acidulated fluorophosphate, amine fluoride, water-soluble salts of fluoride, such as, sodium fluoride, potassium fluoride, calcium fluoride, stannous fluoride, sodium fluorosilicate, bis-salicylato-bis-fluorotitanium (IV), ammonium fluorosilicate, calcium salt, phosphate salt, calcium salt/phosphate salt, calcium salt/ionic fluoride sources, zinc salt/phosphate salt, or any combinations thereof.

Desensitization compounds that can be used in the present whitening strip include, but are not limited to, water-soluble potassium salt including potassium nitrate, potassium citrate, potassium chloride, potassium bicarbonate, potassium oxalate, and tubular occlusion compounds (e.g., ferric oxalate), or any combinations thereof.

Anti-calculus agents that can be used in the present whitening strip include, but are not limited to, one or more phosphates, pyrophosphates, polyphosphates, phosphonates (e.g. ethane-1-hydroxy-1,1-diphosphonate, 1-azacycloheptane-1,1-diphosphonate) polyphosphonates, polyacrylates and other polycarboxylates, ethylenediaminetetraacetic acid and other calcium chelators, carboxylic acids and their salts, zinc salts (e.g. sodium zinc citrate), PVM/MA copolymer or other polymers which interfere with crystal nucleation or growth, or any combinations thereof. It should be understood that when phosphate anti-calculus agent is used in conjunction with phosphate whitening agent, the phosphate anti-calculus agent(s) used is typically different than the phosphate whitening agent(s) used.

Flavoring agents that can be used in the present whitening strip include, but are not limited to, flavoring oil, e.g., oils of spearmint, peppermint, wintergreen, sassafras, clove, sage, eucalyptus, marjoram, cinnamon, lemon, menthol, anethole, thymol, parsley oil, oxanone and orange, alpha-irisone, cassia, marjoram, propenyl guaethol, and methyl salicylate. Sweetening agents including, but not limited to, sucrose, lactose, maltose, sorbitol, xylitol, sodium cyclamate, sucralose, acesulfame-K, aspartame, and sodium saccharin. Any combinations of the preceding flavoring agents are also suitable for use in the whitening strip.

Anti-inflammatory agents that can be used in the present whitening strip include, but are not limited to, non-steroidal anti-inflammatory agents, such as, ketorolac, flurbiprofen, ibuprofen, naproxen, indomethacin, aspirin, ketoprofen, piroxicam, meclofenamic acid, or any combinations thereof. Also, steroidal and non-steroidal anti-inflammatory agents and plant extracts that have demonstrated anti-inflammatory activities can be used.

Antioxidants that can be used in the present whitening strip include, but are not limited to, Vitamin E, ascorbic acid, Uric acid, kojic acid, coenzyme compound (e.g. coenzyme Q-10), carotenoid, Vitamin A, flavonoid and polyphenol, herbal antioxidant, melatonin, aminoindole, lipoic acid, or any combinations thereof.

Other suitable antioxidants include, but are not limited to, rosemary extract, tocopherol, a derivative of tocopherol including a tocotriene, carotene, a carotenoid, a phenolic antioxidant including a phenolic acid, a bioflavonoid, a plant extract, curcumin, tetrahydrocurcumin, camphorol, quercetine, epigenine, or any combinations thereof.

Vitamins that can be used in the present whitening strip include, but are not limited to, Vitamin K, retinol (vitamin A), tocopherol, or any combinations thereof.

In operation, upon contact with the teeth and moisture, the strip, due to the polymer system, hydrates in such a manner as to conform to the contours of the teeth and the interstices, filling any space it encounters. As a result, the interaction of the whitening composition with the surface of the teeth and the interstices between the teeth becomes more intimate and stronger over time. It has been surprisingly found that, following initial application of the strip, the adhesive properties of the strip continue to improve thereby providing better, intimate contact of the strip with and adhesion to the morphology of the teeth.

It should be noted that unlike the strips described in the prior art, the whitening strip of the present invention does not require a backing layer. In fact, in an embodiment of the present invention, the whitening strip does not have a backing layer.

The process for producing a foamed extruded film or sheet may involve three general steps: (1) preparation of the ingredients (including active polymers) in a viscous liquid state, (2) introduction of fine bubbles of gas to produce expansion, and (3) solidification of the foamed plastic film or sheet to stabilize the foamed structure. For thermoplastic materials, the first step can be accomplished by hot melting or heating. For thermosetting materials, they can be prepared by partial pre-polymerization to a viscous liquid intermediate state. In some cases, the solid polymers can be dispersed in a liquid medium to produce the viscous liquid state. The gas used for foaming may be introduced mechanically or chemically. By way of example, air can be introduced mechanically into the process by whipping or beating.

Alternatively, chemical production of gases to cause foaming is most often accomplished by thermal decomposition of organic azo compounds, which liberate nitrogen gas upon heating. Carbon dioxide is often produced by decomposition of carbonates, or by the reaction of some reactive chemicals with water.

It is generally desirable to introduce the gas in fine bubbles to produce a fine cell structure. After foaming of the viscous liquid polymer, it may be solidified to stabilize the foam structure that has been produced. Thermoplastic materials can be solidified by cooling. Sometimes, endothermic evaporation of gases during foaming may be used to aid the cooling process. Thermosetting polymers can be cross-linked and cured during foaming, and thus brought to the final solid state.

The above describes the invention including preferred embodiments thereof. It should be understood that modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

We claim:

1. A transmucosal foamed substrate composition comprising:
one or more polymers; and
one or more foaming agents,
wherein the foamed substrate composition is extrudable into a foamed substrate, and
wherein said foamed substrate provides a cushioning effect to the user during use.

2. The transmucosal foamed substrate of claim 1, wherein said substrate is selected from the group consisting of a film, sheet, strip, wafer and a rope- or string-like form.

3. The transmucosal foamed substrate of claim 1, wherein said substrate is selected from the group consisting of adhesive denture liner, tooth whitening strip, and smoking control/cessation strip.

4. The transmucosal foamed substrate of claim 1, further comprising one or more components selected from the group consisting of plasticizers, hydrophobic barrier agents, tooth whitening agents, antioxidants, preservatives, super-disintegrants or absorbents, flavorants, deodorants, breath freshening agents, colorants, surfactants, film modifiers, cross-linking agents, antimicrobial agents, control release agents, other therapeutic agents, and any combinations thereof.

5. A denture adhesive liner composition comprising:
a denture adhesive effective amount of one or more denture adhesive materials;
one or more thermoplastic polymers;
one or more plasticizers; and
one or more foaming agents,
wherein said composition is extrudable into a foamed denture adhesive liner in the form of a foamed film or foamed sheet that is capable of adhering to a wet mucous surface, and
wherein the foamed denture adhesive liner provides a cushioning effect between a denture and a user's mouth.

6. The denture adhesive liner composition according to claim 5, wherein said one or more denture adhesive materials is selected from the group consisting of polyvinyl alcohols, polyethylene oxides, karaya gums, methyl vinyl ether/maleic anhydride copolymers, hydrated derivatives of methyl vinyl ether/maleic anhydride copolymers, partial salts of methyl vinyl ether/maleic anhydride copolymers, sodium alginate, and any combinations thereof.

7. The denture adhesive liner composition according to claim 5, wherein said one or more thermoplastic polymers is selected from the group consisting of polyethylene oxide polymer, hydroxypropylcellulose, hydroxypropylmethyl cellulose, and any combinations thereof.

8. The denture adhesive liner composition according to claim 5, wherein said one or more plasticizers is selected from the group consisting of water, polyethylene oxide, polypropylene oxide, glycol, propylene glycol, polyethylene glycol, polyhydric alcohol, glycerin, sorbitol, glycerol ester, glycerol triacetate, fatty acid triglyceride, naphthenic oil, aromatic oil, vegetable oil, castor oil, low molecular weight rosin ester, polyterpene, and any combinations thereof.

9. The denture adhesive liner composition according to claim 5, further comprising one or more components selected from the group consisting of hydrophobic barrier agents, antioxidants, preservatives, super-disintegrants, absorbents, flavorants, deodorants, breath freshening agents, colorants, surfactants, film modifiers, cross-linking agents, antimicrobial agents, control release agents, therapeutic agents, and any combinations thereof.

10. A tooth whitening strip composition comprising:
one or more whitening agents;
a water-soluble or water-dispersible polymer system; and
one or more foaming agents,
wherein said tooth whitening strip composition is extrudable into a foamed tooth whitening strip in the form of a foamed film or foamed sheet that is capable of dissolving in an oral environment, and
wherein the foamed tooth whitening strip provides a cushioning effect between a user's teeth and/or gums and mouth.

11. The tooth whitening strip composition of claim 10, wherein said one or more whitening agents are selected from the group consisting of hydrogen peroxide, carbamide peroxide, peroxycarbamate, persulfate, persulfate salt, percarbonate salt, perboric acid, perborate salt, PVP-hydrogen peroxide complex, calcium peroxide, metal chlorite, calcium chlorite, barium chlorite, magnesium chlorite, lithium chlorite, sodium chlorite, potassium chlorite, hydroperoxide, peroxyacid, organic peroxide, benzoyl peroxide, chlorine dioxide, hydrogen peroxide adduct of carbodiimide persulfate, peroxide-generating compounds, azobisisobutyronitrile, phosphate, polyphosphate, alkali metal phosphate, alkali metal polyphosphate, sodium tripolyphosphate, and any combinations thereof.

12. The tooth whitening strip composition of claim 10, wherein said water-soluble or water-dispersible polymer system comprises a water-soluble or water dispersible poly-(vinylpyrrolidone) and at least one second polymer.

13. The tooth whitening strip composition of claim 12, wherein said at least one second polymer is selected from the group consisting of an alkyl vinyl ether/maleic anhydride copolymer, alkyl vinyl ether/maleic acid copolymer, alkali metal or an amine salt of alkyl vinyl ether/maleic acid copolymer, partially or fully cross-linked alkyl vinyl ether/maleic anhydride copolymer, vinyl acetate copolymer, polyacrylates, polyurethane interpolymer, chitosan, poly(acrylic acid), poly(vinyl alcohol), poly(vinyl alcohol-g-ethylene glycol) copolymer, cellulose derivative, hydroxypropyl-methyl cellulose, hydroxylethyl cellulose, hydroxypropyl cellulose, poly(ethylene oxide), poly(propylene oxide), Polyquaterium-11, Polyquaterium-39, poloxamer, carbomer, gelatin, starch, alginic acid, salt of alginic acid, karaya gum, xanthan gum, guar gum, arabic gum tragacanth, and any combinations thereof.

14. The tooth whitening strip composition of claim 12, wherein said water-soluble or water dispersible poly-(vinylpyrrolidone)(PVP) and at least one second polymer are selected from the group consisting of poly(vinylpyrrolidone)-alkyl vinyl ether/maleic anhydride copolymer, poly(vinylpyrrolidone)-alkyl vinyl ether/maleic acid copolymer, poly(vinylpyrrolidone)-alkali metal or an amine salt of alkyl vinyl ether/maleic acid copolymer, poly(vinylpyrrolidone)-partially or fully cross-linked alkyl vinyl ether/maleic anhydride copolymer, poly(vinylpyrrolidone)-vinyl acetate copolymer, poly(vinylpyrrolidone)-polyurethane interpolymer, poly(vinylpyrrolidone)-chitosan, poly(vinylpyrrolidone)-polyacrylates, poly(vinylpyrrolidone)-poly(acrylic acid), poly(vinylpyrrolidone)-poly(vinyl alcohol), poly(vinylpyrrolidone)-poly(vinyl alcohol-g-ethylene glycol) copolymer, poly(vinylpyrrolidone)-cellulose derivatives, poly(vinylpyrrolidone)-hydroxypropyl-methyl cellulose, poly(vinylpyrrolidone)-hydroxyethyl cellulose, poly(vinylpyrrolidone)-hydroxypropyl cellulose, poly(vinyl pyrrolidone)-poly(ethylene oxide), poly(vinylpyrrolidone)-poly(propylene oxide), poly(vinyl pyrrolidone)-Polyquaterium-11, poly(vinylpyrrolidone)-Polyquaterium-39, poly(vinylpyrrolidone)-poloxamer, poly(vinylpyrrolidone)-carbomer, poly(vinylpyrrolidone)-gelatin, poly(vinylpyrrolidone)-starch, poly(vinylpyrrolidone)-alginic acid, poly(vinylpyrrolidone)-salt of alginic acid, poly(vinylpyrrolidone)-gum karaya, poly(vinylpyrrolidone)-xanthan gum, poly(vinylpyrrolidone)-guar gum, poly(vinylpyrrolidone)-arabic gum, poly(vinylpyrrolidone)-tragacanth, and any combinations thereof.

15. The tooth whitening strip composition of claim 10, further comprising one or more components selected from the group consisting of antimicrobial agent, mineralization compound, stain prevention compound, desensitization compound, anti-calculus agent, flavoring agent, anti-inflammatory agent, antioxidant, volatile sulfur scavenger, odorant neutralizer, vitamin, penetration enhancer, plasticizer, preservative, surfactant, wetting agent, anesthetic, anti-allergenic, pharmaceutical, and any combinations thereof.

16. A process for producing a transmucosal foamed substrate comprising the steps of:
preparing a substrate composition in a viscous liquid state;
aerating said substrate composition to produce a foamed composition; and
solidifying said foamed composition thereby forming said transmucosal foamed substrate.

17. The process of claim 16, wherein the step of preparing said substrate composition in a viscous liquid state comprises hot melting or heating.

18. The process of claim 16, wherein the step of preparing said substrate composition in a viscous liquid state comprises partial pre-polymerization of said composition to a viscous liquid intermediate state.

19. The process of claim 16, wherein said aerating step is mechanical aerating, chemical aerating, or any combination thereof.

20. The process of claim 19, wherein said mechanical aerating includes whipping, beating, or any combination thereof.

21. The process of claim 19, wherein said chemical aerating includes a thermal decomposition of one or more organic azo compounds, a decomposition of one or more carbonates, a reaction of one or more reactive chemicals with water, or any combinations thereof.

22. The process of claim 16, wherein said solidifying step comprises cooling, cross-linking, curing, or any combinations thereof.

23. The process of claim 16, wherein said transmucosal foamed substrate is a foamed denture adhesive liner.

24. The process of claim 16, wherein said transmucosal foamed substrate is a foamed tooth whitening strip.
